(19) United States
(12) Patent Application Publication
(10) Pub. No.: US 2025/0092535 A1
(43) Pub. Date: Mar. 20, 2025

(54) LOW ENERGY CONSUMPTION ELECTROCATALYTIC METHOD FOR ELECTROSYNTHESIS OF HYDROGEN PEROXIDE COUPLED WITH OXIDATION UPCYCLING OF PET PLASTICS

(71) Applicant: Beijing University of Chemical Technology, Beijing (CN)

(72) Inventors: Jieshan QIU, Beijing (CN); Jun QI, Beijing (CN); Qi YANG, Beijing (CN)

(73) Assignee: Beijing University of Chemical Technology, Beijing (CN)

(21) Appl. No.: 18/829,262

(22) Filed: Sep. 9, 2024

(30) Foreign Application Priority Data
Sep. 14, 2023 (CN) 202311185025.9

(51) Int. Cl. C25B 1/30; C08J 11/14; C25B 3/07; C25B 3/23; C25B 11/051; C25B 15/08

(52) U.S. Cl. CPC C25B 1/30; C08J 11/14; C25B 3/07; C25B 3/23; C25B 11/051; C25B 15/083

(57) ABSTRACT

A low-energy consumption electrocatalytic method for electrosynthesis of hydrogen peroxide coupled with oxidation upcycling of PET plastics is provided. The method includes ball-milling a waste polyethylene terephthalate (PET) plastic into powder, depolymerizing in alkali solution, and then using the transition metal catalyst and carbon-based material as anode and cathode in the circulating electrolytic cell, under the applied voltage, the cathode undergoes an oxygen reduction reaction to generate hydrogen peroxide, and the anode undergoes an oxidation reaction to upgrade ethylene glycol to formic acid. Products of terephthalic acid and potassium diformate are obtained by adjusting the pH and vacuum distillation of the anode electrolyte. The sodium perborate product or benzoyl peroxide product can be prepared in the cathode electrolyte. The process has significant energy saving effect, avoids the separation of thermodynamically unstable hydrogen peroxide, and simultaneously realizes the recycling of waste PET plastics and the green electrosynthesis of hydrogen peroxide.

### CROSS REFERENCE TO THE RELATED APPLICATIONS

This application is based upon and claims priority to Chinese Patent Application No. 202311185025.9, filed on Sep. 14, 2023, the entire contents of which are incorporated herein by reference.

### TECHNICAL FIELD

The present invention belongs to the field of electrosynthesis and electrocatalysis, and specifically relates to a low-energy consumption electrocatalytic method for electrosynthesis of hydrogen peroxide coupled with oxidation upcycling of PET plastics.

### BACKGROUND

Hydrogen peroxide (H₂O₂) is an important chemical reagent, it is widely used in sterilization, textile bleaching, wastewater treatment, semiconductor chip cleaning, and industrial waste treatment thanks to its excellent oxidation capacity. At present, the conventional anthraquinone method is mainly used for large-scale preparation in the industry, but the anthraquinone process requires a large amount of H₂, produces a large amount of organic waste, and requires a complex separation process to obtain high-purity H₂O₂ that meets the market demand. In addition, this process requires centralized large-scale infrastructure to be implemented, the transportation cost of high-concentration H₂O₂ is high and there are safety risks. In contrast, the preparation of H₂O₂ by electrosynthesis process driven by secondary energy such as wind energy and solar energy has the advantages of simple and controllable device, low fixed cost, low energy consumption, and low waste emission, which has the characteristics of safety, simplicity, green and low carbon, and presents a broad development prospect.

As an important synthetic product, plastic has developed rapidly in recent years, global primary plastic production will reach an astonishing 34 billion tons by 2050 according to data released by the United Nations Environment Programme (UNEP), considering the current level of plastic waste treatment, how strengthening the recycling and management of plastic waste has become a global urgent problem, according to statistics, less than 10% of the billions of tons of plastic waste generated worldwide has been recycled, and the value loss associated with plastic waste is 80 billion dollars to 120 billion dollars per year. The damage to the environment and soil caused by conventional incineration and landfills is also inevitable, so chemical recovery provides new options, especially the upcycling of electrochemical recovery, the advantages of electrochemical recovery are more prominent with the gradual reduction of power cost. Chinese invention patent CN113774399A discloses a method for co-production of hydrogen, formic acid and terephthalic acid by electrocatalysis of waste PET plastics, hydrogen is produced by the reduction of water in the aqueous electrolyte at the cathode, and ethylene glycol is oxidized at the anode to form formic acid, which simultaneously realizes the recovery of plastics and green hydrogen production. Chinese invention patent CN113502493A discloses a system and method for photoelectrocatalytic oxidation of organic solid waste coupled with carbon dioxide reduction, which improves the rate of photoelectrocatalytic carbon dioxide reduction.

At present, the electrochemical recovery of PET plastics mainly focuses on oxidation upcycling of PET coupled with green hydrogen production, for the electrosynthesis of hydrogen peroxide, mainly focuses on the design of the catalyst and the revelation of the mechanism, the corresponding anode reaction is an oxygen evolution reaction, which has high energy consumption, and the value of oxygen as an anode reaction product is low, compared with the conventional anthraquinone method, it has no obvious advantages in terms of cost and benefit. Currently, there is no research report on the electrolysis system of electrochemical oxidation upcycling of PET plastic coupled with cathodic oxygen reduction to produce hydrogen peroxide, and there are still gaps from the design of catalysts to the construction of electrolysis devices, and the separation and recovery of products, in particular, the instability of cathodic hydrogen peroxide in alkaline medium greatly limits the separation and purification of products.

### SUMMARY

In order to overcome the shortcomings of the existing technology, a low energy consumption electrocatalytic method for electrosynthesis of hydrogen peroxide coupled with oxidation upcycling of PET plastics is proposed, the process has strong innovation, lower energy consumption than conventional electrosynthesis of hydrogen peroxide, and realizes the conversion of anode waste to high value-added products, which alleviates the plastic crisis and provides a new feasible path for electrosynthesis of hydrogen peroxide.

In order to realize the above objective of the present invention and solve the problems existing in the existing technology, the technical scheme adopted by the present invention is: a low energy consumption electrocatalytic method for electrosynthesis of hydrogen peroxide coupled with oxidation upcycling of PET plastics, comprising the following steps:
(1) ball-milling a waste polyethylene terephthalate plastic into powder, then adding the power into an alkali solution, heating in a water bath and stirring at a high speed to depolymerize into a monomer solution of terephthalic acid and ethylene glycol;
(2) in the circulating electrolytic cell, using the monomer solution as an electrolyte at an anode, using a sodium hydroxide solution as the electrolyte at a cathode, separating a cathode chamber and an anode chamber by an anion exchange membrane, the cathode side separates a gas chamber and a liquid chamber through a gas diffusion electrode, and the gas chamber is filled with oxygen, different catalysts are used on the cathode side and anode side respectively, driven by an applied voltage, the cathode undergoes an oxygen reduction reaction to generate hydrogen peroxide, and the anode undergoes electrochemical oxidation of ethylene glycol to formic acid;
(3) after electrolysis, transferring a cathode electrolyte and an anode electrolyte to a separation system through a pump respectively;
separation of an anode product: directly precipitating terephthalic acid contained in the anode electrolyte by adjusting a pH to acidity, and then obtaining a terephthalic acid product by washing and drying, and obtaining a potassium diformate product by vacuum distillation of the remaining electrolyte;
(4) separation of a cathode product comprises two methods:
method 1: placing an electrolyte containing hydrogen peroxide in the cathode into a reactor, and adding a homogeneous aqueous solution containing borax and sodium hydroxide while maintaining the temperature of the ice water bath, after completing a reaction, filtering an obtained precipitate, then washing and drying to obtain a sodium perborate product;
method 2: placing the electrolyte containing hydrogen peroxide in the cathode into the reactor, and adding sodium dodecyl benzene sulfonate as a catalyst, maintaining a temperature of the water bath, and gradually adding benzoyl chloride dropwise, then filtering, washing and drying a crystalline precipitate to obtain a benzoyl peroxide.

In step (1), the alkali solution is a potassium hydroxide solution or a sodium hydroxide solution, a concentration of alkali solution is 0.1-5 mol/L, a temperature of the water bath is 60-100° C., and a rotation speed is greater than 300 r/min.

The selection of cathode catalyst is not the focus of this process, in the existing technology, and heteroatom-doped carbon materials, Co or Pd-based single-atom catalysts, etc. with a Faraday efficiency for hydrogen peroxide in excess of 85% can be applied to this process.

The cathode catalyst is selected from a B/N co-doped carbon black catalyst or a Co single-atom catalyst of Co—N—C structure.

The selection of an anode catalyst is also not the focus of this process, in the existing technology, a transition metal oxide, hybrid and hydrotalcite catalyst with Ni, Co or Cu as a metal center is selected as the anode catalyst, a catalyst with a Faraday efficiency for formate in excess of 90% can be applied to this process, but Pd and Pt noble metal catalysts cannot be used. The anode catalyst is selected from NiCo-LDH/NF or NiSₓ/NF.

In step (2), a reaction temperature of the electrolytic cell is 25-100° C., a pressure is 0.1-1 MPa, the anion exchange membrane is alcohol-resistant, and the electrolyte is circulated in a device by the pump.

In step (3), the anode electrolyte is adjusted to pH 3-4 by formic acid, and the terephthalic acid is precipitated.

In method 1 of step (4), a temperature of the ice water bath is 2-10° C. and a reaction time is 1-10 h when sodium perborate is prepared.

In method 2 of step (4), a temperature of the water bath is 5-15° C. and the reaction time is 1-10 h when benzoyl peroxide is prepared.

Further, the specific steps are:

step 1: ball-milling the waste polyethylene terephthalate (PET) plastic into powder, then adding the power into a 0.1-5 mol/L potassium hydroxide solution, heating to 60-100° C. in the water bath and stirring at high speed to fully depolymerize the PET into the monomer of terephthalic acid (PTA) and ethylene glycol (EG).

Step 2: in the circulating electrolytic cell, using the solution obtained in step 1 as the electrolyte at the anode, using a 0.1-5 mol/L sodium hydroxide solution as the electrolyte at the cathode, separating the cathode chamber and the anode chamber by the anion exchange membrane, using a transition metal-based compound and a carbon-based material as anode and cathode side catalysts, respectively, the cathode side separates the gas chamber and the liquid chamber through the gas diffusion electrode, and the gas chamber is filled with oxygen, driven by the applied voltage, the cathode undergoes the oxygen reduction reaction to generate hydrogen peroxide, and the anode undergoes electrochemical oxidation of EG to formic acid. Using a peristaltic pump to drive the electrolyte to circulate in the device, after the product reaches a certain concentration, transferring it to the separation system for separation.

Step 3: after electrolysis, transferring the cathode electrolyte and the anode electrolyte to the different separation system through a pump respectively, directly precipitating PTA contained in the anode electrolyte by adding formic acid to adjust the pH to 3-4, and then obtaining the PTA product by washing and drying, and obtaining the potassium diformate under the operation of vacuum distillation of the remaining electrolyte, and obtaining the potassium diformate product by subsequent drying treatment.

Step 4: placing the electrolyte containing hydrogen peroxide in the cathode into the reactor, and adding the homogeneous aqueous solution containing borax and sodium hydroxide while maintaining the temperature of the ice water bath to be lower than 10° C., after completing the reaction, filtering the obtained precipitate, then washing and drying to obtain the sodium perborate product; another method is to place the electrolyte containing hydrogen peroxide into the reactor, and adding sodium dodecyl benzene sulfonate as a catalyst, maintaining the temperature of the water bath below 15° C., and gradually adding benzoyl chloride dropwise, then filtering, washing and drying the crystalline precipitate to obtain the benzoyl peroxide product that meets the demand.

The beneficial effect of the present invention is that the waste polyethylene terephthalate (PET) plastic is ball-milled into powder and depolymerized in alkali solution, and then the transition metal catalyst and carbon-based material are used as anode and cathode in the circulating electrolytic cell, under the applied voltage, the cathode undergoes oxygen reduction reaction to generate hydrogen peroxide, and the anode undergoes oxidation reaction to upgrade ethylene glycol to formic acid. Terephthalic acid and potassium diformate products are obtained by adjusting pH and vacuum distillation of the anode electrolyte; the sodium perborate product or benzoyl peroxide product can be prepared in the cathode electrolyte.

In terms of energy saving, this innovative coupling method of hydrogen peroxide synthesis and PET recovery is more energy-efficient than the conventional hydrogen peroxide electrosynthesis coupled with oxygen evolution system, because EG monomer has a lower onset potential and operating potential than oxygen evolution in thermodynamics; in addition, while realizing the electrosynthesis of hydrogen peroxide, the plastic crisis can be alleviated, and the waste PET plastic can be upgraded to PTA and potassium diformate products with high added value, which improves the market competitiveness of the whole process (FIG. 1). In general, the electrosynthesis of hydrogen peroxide coupled with oxidation upcycling of PET plastics process disclosed by the present invention has never been reported before, and has very attractive economic potential and application value, which opens up a new path for electrosynthesis of hydrogen peroxide and recycling of waste plastics, and has broad application prospects.

### BRIEF DESCRIPTION OF THE DRAWINGS

FIG. 1 is a schematic diagram of an electrosynthesis of hydrogen peroxide coupled with oxidation upcycling of PET plastics process in embodiment 1.

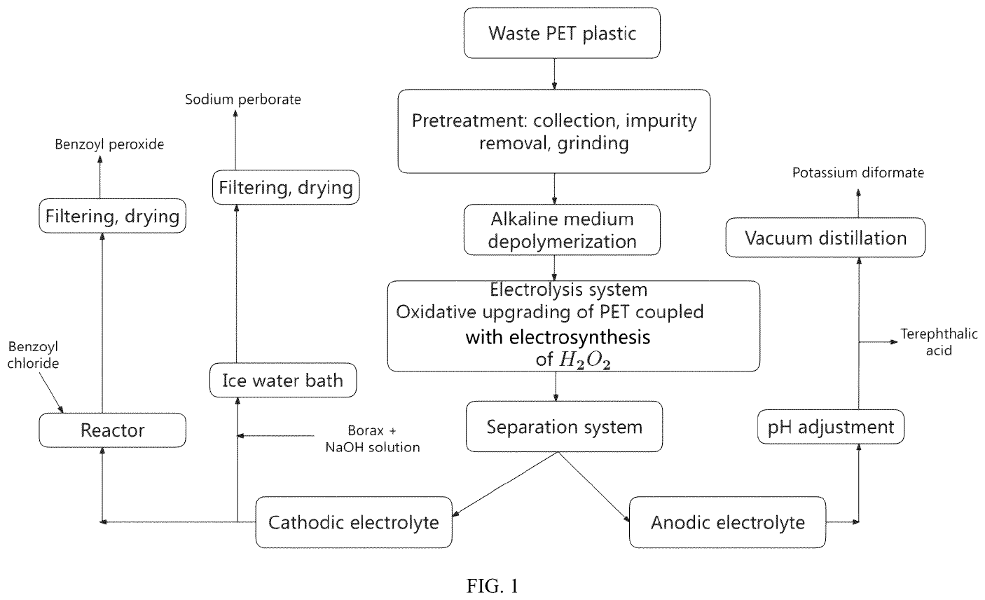

FIG. 2 is a polarization curve of an electrosynthesized hydrogen peroxide coupled with oxidation system of PET plastics in embodiment 1.

FIG. 3 is an XRD pattern of a potassium diformate product in embodiment 2.

FIG. 4 is an XRD pattern of a sodium perborate product in embodiment 2.

FIG. 5 is an XRD pattern of a benzoyl peroxide product in embodiment 2.

### DETAILED DESCRIPTION OF THE EMBODIMENTS

The following is a further explanation of the present invention in combination with embodiments. The following non-restrictive implementation measures can enable ordinary technicians in this field to understand the present invention more comprehensively, but do not limit the present invention in any way.

Unless otherwise specified, the experimental methods described in the following examples are conventional; unless otherwise specified, reagents and materials are all commercially available.

### Embodiment 1

The exploration of PET plastic depolymerization conditions in step 1 of the electrosynthesis of hydrogen peroxide coupled with oxidation upcycling of PET plastics process is as follows:

the waste polyethylene terephthalate (PET) plastic is ball-milled into powder, then the power is added into the potassium hydroxide solution with different concentrations (0.1 mol/L, 0.5 mol/L, 1 mol/L, 5 mol/L), heated to 70° C. in the water bath and stirred at the high speed for 4 h to fully depolymerize the PET into the monomer of terephthalic acid (PTA) and ethylene glycol (EG). Then, the concentration of PTA and EG in the depolymerization solution is determined by 1H NMR, the results showed that with the increase of potassium hydroxide concentration, the depolymerization is more sufficient, and the concentration of monomer in the solution is higher, however, the alkali resistance of the subsequent electrolysis device needs to be considered, the excessive alkali concentration will accelerate the corrosion of the device, therefore, the relatively reasonable concentration of potassium hydroxide solution can be selected 1-2 mol/L.

Subsequently, the depolymerization temperature is explored (60° C., 70° C., 80° C., 90° C., and 100° C.), similar to the concentration of alkali solution, with the increase of temperature, the depolymerization is easier, but the high temperature will cause damage to the undepolymerized PET plastic powder, and more energy is needed, the more reasonable depolymerization temperature is 70-80° C.

For the speed mentioned in the depolymerization conditions, the increase of the speed is beneficial to the depolymerization of PET powder, and the more reasonable rotation speed of depolymerization is greater than 300 r/min.

### Embodiment 2

The exploration of the anode catalyst in step 1 of the electrosynthesis of hydrogen peroxide coupled with oxidation upcycling of PET plastics process is as follows:

step 1, the waste polyethylene terephthalate (PET) plastic is ball-milled into powder, then the power is added into to the 1 mol/L potassium hydroxide solution, heated to 70° C. in the water bath and stirred at high speed for 4 h to fully depolymerize the PET into the monomer of terephthalic acid (PTA) and ethylene glycol (EG).

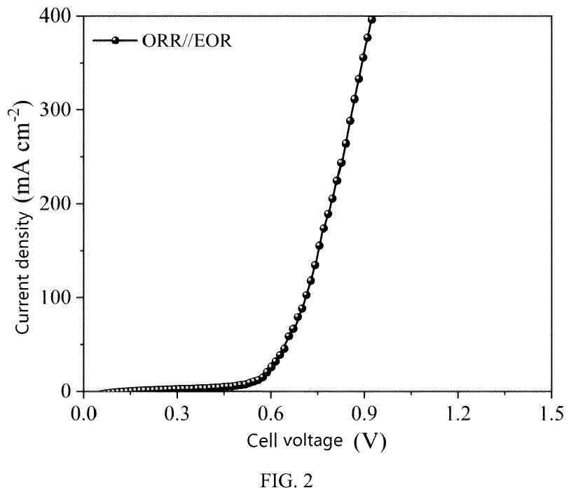

Step 2, according to the method reported in the literature, the anode catalyst NiCo-LDH/NF with high activity and stability is synthesized, specifically: the precursor of Ni salt and Co salt is added to the beaker according to the molar ratio of 9:1, and then 0.5 g urea and 30 mL water are added to dissolve ultrasonically, and the above solution is placed in a 50 mL high-pressure hydrothermal reaction kettle with the pretreated 2*4 cm² nickel foam at 120° C. for 12 h, after cooling, it is taken out and rinsed with ethanol and water, and dried in an oven overnight to obtain NiCo-LDH/NF catalyst and directly used the NiCo-LDH/NF catalyst as the anode catalyst without the use of binders. Meanwhile, heteroatom-doped carbon-based catalysts are employed as the cathode to perform electrochemical performance tests in a circulating electrolytic cell, the solution obtained in step 1 is used as the electrolyte at the anode, the 1 mol/L sodium hydroxide solution is used as the electrolyte at the cathode, the cathode chamber and the anode chamber are separated by the anion exchange membrane, the cathode side separates the gas chamber and the liquid chamber through the gas diffusion electrode, and the gas chamber is filled with oxygen, driven by the applied voltage, the cathode undergoes the oxygen reduction reaction to generate H₂O₂, and the anode undergoes electrochemical oxidation of EG to formic acid. Chenhua 760E workstation is used to evaluate the electrochemical performance, according to the LSV curve of the two-electrode system, it can be found that the cell voltage required for the electrosynthesis of hydrogen peroxide coupled with oxidation upcycling of PET plastics process to achieve an industrial current density of 400 mA cm⁻² is only 0.93 V (FIG. 2), which has excellent energy saving effect, and the anode NiCo-LDH/NF also shows excellent catalytic capacity and selectivity for the oxidation of EG monomer, in which the Faraday efficiency of formate exceeds 95%.

### Embodiment 3

The exploration of the anode catalyst in step 1 of the electrosynthesis of hydrogen peroxide coupled with oxidation upcycling of PET plastics process is as follows:

step 1, the waste polyethylene terephthalate (PET) plastic is ball-milled into powder, then the power is added into to the 1 mol/L potassium hydroxide solution, heated to 70° C. in the water bath and stirred at high speed for 4 h to fully depolymerize the PET into the monomer of terephthalic acid (PTA) and ethylene glycol (EG).

Step 2, according to the method reported in the literature, the anode catalyst NiSₓ/NF with high activity and stability is synthesized, specifically: the commercial nickel foam is ultrasonically treated in dilute hydrochloric acid for 30 min, and then washed with ethanol and water to remove the oxide layer on the surface, subsequently, 0.5 mol/L sodium chloride aqueous solution containing 1 mmol Ni(NO₃)₂·6H₂O is prepared, the nickel foam is used as the working electrode, the Ag/AgCl electrode and the carbon rod are used as the reference electrode and the counter electrode for electrochemical deposition, and then the Ni(OH)₂/NF nanosheets immobilized on the nickel foam skeleton are prepared, then it is transferred to 50 ml high-pressure hydrothermal reaction kettle, and meanwhile ammonium thiosulfate ethanol solution is added and kept at 120° C. for 3 h, and liquid phase sulfurization is carried out, after cooling, it is taken out and rinsed with ethanol and water, and dried in an oven overnight to obtain NiSₓ/NF catalyst and directly used the NiSₓ/NF catalyst as an anode catalyst. Similar to step 2, heteroatom-doped carbon-based catalysts are employed as the cathode to perform electrochemical performance tests in a circulating electrolytic cell, the cathode chamber and the anode chamber are separated by the anion exchange membrane, the cathode side separates the gas chamber and the liquid chamber through the gas diffusion electrode, and driven by the applied voltage, the cathode undergoes the oxygen reduction reaction to generate hydrogen peroxide, and the anode undergoes electrochemical oxidation of EG to formic acid. Chenhua 760E workstation is used to evaluate the electrochemical performance, according to the LSV curve of the two-electrode system, it can be found that the cell voltage required for the electrosynthesis of hydrogen peroxide coupled with oxidation upcycling of PET plastics process to achieve an industrial current density of 400 mA cm⁻² is only 0.96, moreover, the anode NiSₓ/NF also showed excellent catalytic capacity and selectivity for the oxidation of EG monomer, and the Faraday efficiency of formate exceeds 93%.

### Embodiment 4

The exploration of the cathode catalyst in step 1 of the electrosynthesis of hydrogen peroxide coupled with oxidation upcycling of PET plastics process is as follows:

step 1, the waste polyethylene terephthalate (PET) plastic is ball-milled into powder, then the power is added into to the 0.1-5 mol/L potassium hydroxide solution, heated to 60-100° C. in the water bath and stirred at the high speed to fully depolymerize the PET into the monomer of terephthalic acid (PTA) and ethylene glycol (EG).

Step 2, the B/N co-doped carbon black catalyst is prepared as the cathode catalyst, and 10 g of commercially purchased carbon black is weighed and placed in an oxygen plasma machine to treat the surface functional groups of carbon black in an oxygen-rich environment. According to the mass ratio of 1:5 ratio to weigh the treated carbon black 2 g and boric acid 10 g and added into a mortar for grinding, so that the boric acid are fully mixed with the carbon black. After grinding, the mixture is placed in a porcelain boat and then placed in the middle of a tube furnace, the heating program is set at 5° C./min, and the temperature is raised to 800° C. for 2 h, then cooled and the sintered mixture powder is taken out. The mixture powder is put into deionized water, and then placed in a water bath environment heated to 80° C. for 4 h, in order to fully remove the mixed boron oxide impurities, and then filtered and dried, and used as the catalyst for future use. According to the electrochemical test procedure of step 2, the B/N co-doped carbon black is used as the cathode catalyst in the circulating electrolytic cell, and configured as ink droplets to coat on the gas diffusion electrode, the solution obtained in step 1 is used as the electrolyte at the anode, and the 2 mol/L sodium hydroxide solution is used as the electrolyte at the cathode, the cathode chamber and the anode chamber are separated by the anion exchange membrane, NiCo-LDH/NF is directly used as the anode catalyst, the cathode side separates the gas chamber and the liquid chamber through the gas diffusion electrode, and driven by the applied voltage, the cathode undergoes the oxygen reduction reaction to generate hydrogen peroxide, and the anode undergoes an electrochemical oxidation of EG to formic acid. Chenhua 760E workstation is used to evaluate the electrochemical performance, the results show that the B/N co-doped carbon black has good catalytic performance for the preparation of hydrogen peroxide by cathodic oxygen reduction, wherein, the industrial current density of 400 mA cm⁻² is only 0.6 V (vs.RHE), and according to the analysis results, the Faraday efficiency of hydrogen peroxide exceeds 97% and reaches more than 90% in a wide voltage range.

### Embodiment 5

The exploration of the cathode catalyst in step 1 of the electrosynthesis of hydrogen peroxide coupled with oxidation upcycling of PET plastics process is as follows:

step 1, the waste polyethylene terephthalate (PET) plastic is ball-milled into powder, then the power is added into to the 0.1-5 mol/L potassium hydroxide solution, heated to 60-100° C. in the water bath and stirred at the high speed to fully depolymerize the PET into the monomer of terephthalic acid (PTA) and ethylene glycol (EG).

Step 2, the Co single-atom catalyst of Co—N—C structure is prepared as the cathode catalyst, the specific steps involve: firstly, the ZIF-67 catalyst is prepared by using dimethylimidazole and Co(NO₃)₂·6H₂O according to the method reported in the literature, and then placed in the middle of the tube furnace, in the ammonia atmosphere or nitrogen atmosphere, the heating program is set to 5° C./min, heated to 800° C. for 2 h, and then cooled as a catalyst for future use. According to the electrochemical test procedure of step 2, the Co single-atom catalyst is used as the cathode catalyst in the circulating electrolytic cell, and configured as ink droplets to coat on the gas diffusion electrode, the solution obtained in step 1 is used as the electrolyte at the anode, and the 2 mol/L sodium hydroxide solution is used as the electrolyte at the cathode, the cathode chamber and the anode chamber are separated by the anion exchange membrane, NiCo-LDH/NF is directly used as the anode catalyst, the cathode side separates the gas chamber and the liquid chamber through the gas diffusion electrode, and driven by the applied voltage, the cathode undergoes the oxygen reduction reaction to generate hydrogen peroxide, and the anode undergoes an electrochemical oxidation of EG to formic acid. Chenhua 760E workstation is used to evaluate the electrochemical performance, the results show that the Co single-atom catalyst also has good catalytic performance for the preparation of H₂O₂ by cathodic oxygen reduction, wherein, the current density of 200 mA cm⁻² is only 0.65 V (vs.RHE), and according to the analysis results, the Faraday efficiency of hydrogen peroxide exceeds 93.5% and reaches more than 88% in a wide voltage range.

### Embodiment 6

The exploration of the reaction conditions of the electrolysis device in step 1 of the electrosynthesis of hydrogen peroxide coupled with oxidation upcycling of PET plastics process is as follows:

step 1, the waste polyethylene terephthalate (PET) plastic is ball-milled into powder, then the power is added into to the 1 mol/L potassium hydroxide solution, heated to 70° C. in the water bath and stirred at high speed to fully depolymerize the PET into the monomer of terephthalic acid (PTA) and ethylene glycol (EG).

Step 2, in the circulating electrolytic cell, the solution obtained in step 1 is used as the electrolyte at the anode, the 1 mol/L sodium hydroxide solution is used as the electrolyte at the cathode, the cathode chamber and the anode chamber are separated by the anion exchange membrane, NiCo-LDH/NF and B/N co-doped carbon black are used as anode and cathode side catalysts, respectively, the cathode side separates the gas chamber and the liquid chamber through the gas diffusion electrode, and the gas chamber is filled with oxygen, driven by the applied voltage, the cathode undergoes the oxygen reduction reaction to generate hydrogen peroxide, and the anode undergoes an electrochemical oxidation of EG to formic acid. The catalyst is pre-activated by CV scanning using a Chenhua 760E electrochemical workstation, and then replaced with a two-electrode system for polarization curve LSV scanning, the scanning rate is 20 mV/s, the scanning range is 0-1.5 V, and the iR compensation is set to 100%, and continuous scanning multiple times until it tends to be stable. The electrolysis device simulates a commercial electrolytic water device, and the reaction temperature control variables are 25° C., 50° C., 65° C., 85° C., and 100° C. The pressure is set to 0.1 MPa, 0.5 MPa and 1 MPa by adjusting the variables. The peristaltic pump is used to drive the electrolyte to circulate in the device, after the product reaches a certain concentration, it is transferred to the separation system for separation.

Electrochemical tests are carried out under different operating conditions of electrolytic devices, it is found that the temperature of the electrolytic device had a significant effect on the catalytic activity and catalytic rate through the test of the LSV curve and the quantitative analysis of the products, with the increase of temperature, the transfer rate of electrons and ions is accelerated, and the catalytic reaction rate is also significantly improved, however, considering the energy consumption required for high temperature and the evaporation rate of water vapor, the temperature can usually be set at 65-85° C. The effect of pressure on the electrolysis device is not obvious, and it can be set to 0.1-1 MPa without special requirements.

### Embodiment 7

The exploration of the separation conditions of anode products in step 1 of the electrosynthesis of hydrogen peroxide coupled with oxidation upcycling of PET plastics process is as follows:

step 1, the waste polyethylene terephthalate (PET) plastic is ball-milled into powder, then the power is added into to the 1 mol/L potassium hydroxide solution, heated to 70° C. in the water bath and stirred at high speed to fully depolymerize the PET into the monomer of terephthalic acid (PTA) and ethylene glycol (EG).

Step 2, in the circulating electrolytic cell, the solution obtained in step 1 is used as the electrolyte at the anode, the 1 mol/L sodium hydroxide solution is used as the electrolyte at the cathode, the cathode chamber and the anode chamber are separated by the anion exchange membrane, NiCo-LDH/NF and B/N co-doped carbon black are used as anode and cathode side catalysts, respectively, driven by the applied voltage, the cathode undergoes the oxygen reduction reaction to generate hydrogen peroxide, and the anode undergoes an electrochemical oxidation of EG to formic acid. The peristaltic pump is used to drive the electrolyte to circulate in the device, after the product reaches a certain concentration, it is transferred to the separation system for separation.

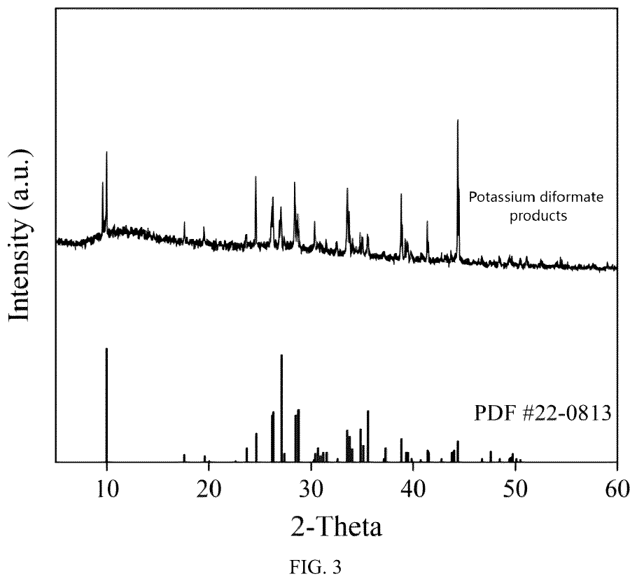

Step 3, the separation of anode products mainly involves the purification of PTA and formate, wherein considering the poor water solubility of PTA in an acidic environment, formic acid can be added to the anode electrolyte from the electrolysis system of step 2 to adjust the pH to 2, 3, 4, 5 and 6, the result shows that PET has different degrees of precipitation in these pH environments, but the precipitation of PAT is not sufficient when the pH is 5 and 6, and the high concentration of acid solution is easy to cause the corrosion of the sedimentation tank after the pH reaches 2, the more reasonable pH adjustment environment is 3-4, in addition, considering the remaining formic acid and potassium formate in the solution, potassium diformate with higher added value can be obtained by vacuum distillation instead of pure potassium formate (FIG. 3), which can further improve the economic profitability of the overall process to a certain extent.

### Embodiment 8

The exploration of the separation conditions of cathode products in step 1 of the electrosynthesis of hydrogen peroxide coupled with oxidation upcycling of PET plastics process is as follows:

step 1, the waste polyethylene terephthalate (PET) plastic is ball-milled into powder, then the power is added into to the 1 mol/L potassium hydroxide solution, heated in the water bath and stirred at high speed to fully depolymerize the PET into the monomer of terephthalic acid (PTA) and ethylene glycol (EG).

Step 2, in the circulating electrolytic cell, NiCo-LDH/NF and B/N co-doped carbon black are used as anode and cathode side catalysts, respectively, driven by the applied voltage, and the electrochemical reaction is carried out, after the product reaches a certain concentration, it transferred to the separation system for separation.

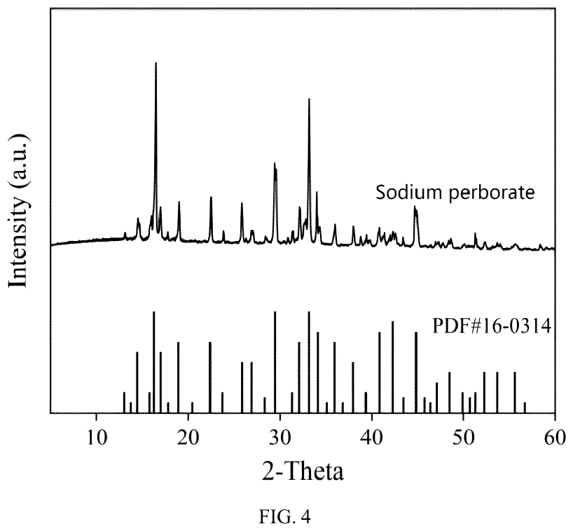

Step 3, the separation of cathode products. Firstly, the borax is dissolved in the aqueous solution containing 1 mol/L sodium hydroxide, heated to 45° C. and fully stirred to filter out the insoluble matter, then, the above solution is added to the reactor, and the ice water bath temperature is maintained at 2° C., 5° C., 10° C. and 15° C., the cathode electrolyte from the electrolysis system is gradually injected, in which the content of H₂O₂ should be more than 5%, as the reaction progresses, white crystals gradually precipitate, that is, sodium perborate, when the reaction temperature is greater than 10° C., the precipitation is not easy to precipitate, the reaction temperature should be strictly controlled during the reaction, after the reaction completed, the precipitate obtained by filtration is washed and dried to obtain the sodium perborate product (FIG. 4).

### Embodiment 9

The exploration of the separation conditions of cathode products in step 1 of the electrosynthesis of hydrogen peroxide coupled with oxidation upcycling of PET plastics process is as follows:

step 1, the waste polyethylene terephthalate (PET) plastic is ball-milled into powder, then the power is added into to the 1 mol/L potassium hydroxide solution, heated in the water bath and stirred at high speed to fully depolymerize the PET into the monomer of terephthalic acid (PTA) and ethylene glycol (EG).

Step 2, in the circulating electrolytic cell, NiCo-LDH/NF and B/N co-doped carbon black are used as anode and cathode side catalysts, respectively, driven by the applied voltage, and the electrochemical reaction is carried out, after the product reaches a certain concentration, it transferred to the separation system for separation.

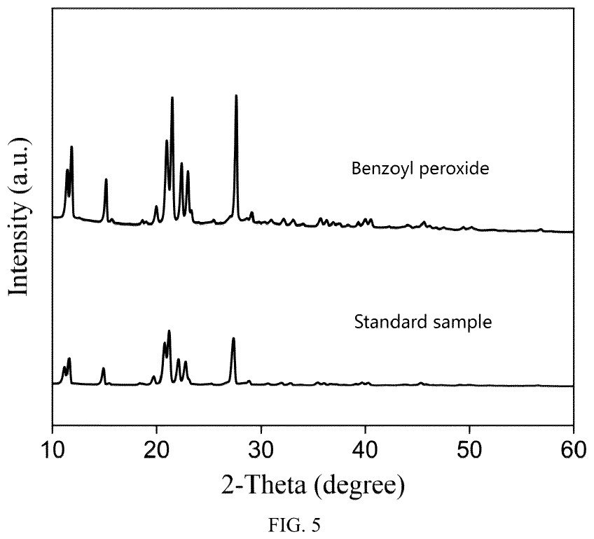

Step 3, the separation of cathode products. Considering the instability of H₂O₂ in alkaline solution, especially during distillation separation, it is easy to cause the decomposition of H₂O₂ and reduce the yield, the H₂O₂ product is innovatively selected to directly produce the downstream product in the present invention, which avoids the limitation of the separation process and the cost of energy consumption, here, it is reacted to produce benzoyl peroxide. firstly, a 500 mL three-necked bottle with stirring is fixed in a constant temperature water bath, the sodium bicarbonate and sodium dodecyl sulfate are dissolved in 50 mL water and added to the reaction bottle, then, benzoyl chloride and cathode containing hydrogen peroxide are added dropwise with the drip filter bucket at the same time, the stirring and dropping speed are controlled, the reaction temperature is set at 5° C., 10° C. and 15° C., after 2 hours of reaction, filtration, water washing, soaking, water washing and suction filtration are performed, and the dryer is cooled and dried to obtain a white powder product benzoyl peroxide (FIG. 5), the reaction temperature should be strictly controlled during the reaction process.

Benzoyl peroxide is the most widely used initiator in polymerization, it is mainly used as an initiator and crosslinking agent for PVC, polyacrylonitrile, acrylate, chloroprene rubber, SBS and methyl methacrylate graft polymerization, unsaturated polyester resin curing, organic glass adhesive and so on. Benzoyl peroxide is used as a curing agent and crosslinking agent of silicone rubber and fluorine rubber in the rubber industry. Sodium perborate is mainly used as an oxidant, disinfectant, bactericide, mordant, deodorizer, electroplating solution additive, etc. The synthesis process and product separation process disclosed in the present invention innovatively avoid the separation of hydrogen peroxide, and prepare chemicals with more economic value, which has broad industrial application prospects and practical application value.

Although the implementation measures of the reference embodiment have shown and described the present invention in detail, ordinary technicians in this field should understand that without violating the spirit and scope of the present invention defined by the claim, various forms and details can be changed in it, and various implementation schemes can be combined.

What is claimed is:

1. A low energy consumption electrocatalytic method for electrosynthesis of hydrogen peroxide coupled with oxidation upcycling of polyethylene terephthalate (PET) plastics, comprising the following steps:
(1) ball-milling a waste polyethylene terephthalate plastic into powder, then adding the power into an alkali solution, heating in a first water bath and stirring at a high speed to depolymerize into a monomer solution of terephthalic acid and ethylene glycol;
(2) in a circulating electrolytic cell, using the monomer solution as an electrolyte at an anode, using a sodium hydroxide solution as an electrolyte at a cathode, separating a cathode chamber and an anode chamber by an anion exchange membrane, wherein a cathode side separates a gas chamber and a liquid chamber through a gas diffusion electrode, and the gas chamber is filled with oxygen, different catalysts are used on the cathode side and an anode side respectively, driven by an applied voltage, the cathode undergoes an oxygen reduction reaction to generate the hydrogen peroxide, and the anode undergoes an electrochemical oxidation of the ethylene glycol to formic acid;
(3) after electrolysis, transferring a cathode electrolyte and an anode electrolyte to a separation system through a pump respectively;
wherein a separation of an anode product comprises: directly precipitating the terephthalic acid contained in the anode electrolyte by adjusting a pH of the anode electrolyte to acidity, and then obtaining a terephthalic acid product by washing and drying, and obtaining a potassium diformate product by vacuum distillation of a remaining electrolyte;
(4) a separation of a cathode product comprises two methods:
method 1: placing an electrolyte containing the hydrogen peroxide in the cathode into a reactor, and adding a homogeneous aqueous solution containing borax and sodium hydroxide while maintaining a temperature of an ice water bath, after completing a reaction, filtering an obtained precipitate, then washing and drying to obtain a sodium perborate product;
method 2: placing the electrolyte containing the hydrogen peroxide in the cathode into the reactor, and adding sodium dodecyl benzene sulfonate as a catalyst, maintaining a temperature of a second water bath, and gradually adding benzoyl chloride dropwise, then filtering, washing, and drying a crystalline precipitate to obtain a benzoyl peroxide.

2. The low energy consumption electrocatalytic method for the electrosynthesis of the hydrogen peroxide coupled with the oxidation upcycling of the PET plastics according to claim 1, wherein in the step (1), the alkali solution is a potassium hydroxide solution or the sodium hydroxide solution, a concentration of the alkali solution is 0.1-5 mol/L, a temperature of the first water bath is 60-100° C., and a rotation speed of the stirring is greater than 300 r/min.

3. The low energy consumption electrocatalytic method for the electrosynthesis of the hydrogen peroxide coupled with the oxidation upcycling of the PET plastics according to claim 1, wherein a cathode catalyst is heteroatom-doped carbon materials or Co or Pd-based single-atom catalysts.

4. The low energy consumption electrocatalytic method for the electrosynthesis of the hydrogen peroxide coupled with the oxidation upcycling of the PET plastics according to claim 3, wherein the cathode catalyst is selected from a B/N co-doped carbon black catalyst or a Co single-atom catalyst of a Co—N—C structure.

5. The low energy consumption electrocatalytic method for the electrosynthesis of the hydrogen peroxide coupled with the oxidation upcycling of the PET plastics according to claim 1, wherein a transition metal oxide, hybrid, and hydrotalcite catalyst with Ni, Co or Cu as a metal center is selected as an anode catalyst, and Pd and Pt noble metal catalysts cannot be used.

6. The low energy consumption electrocatalytic method for the electrosynthesis of the hydrogen peroxide coupled with the oxidation upcycling of the PET plastics according to claim 1, wherein an anode catalyst is selected from NiCo-LDH/NF or NiSₓ/NF.

7. The low energy consumption electrocatalytic method for the electrosynthesis of the hydrogen peroxide coupled with the oxidation upcycling of the PET plastics according to claim 1, wherein in the step (2), a reaction temperature of the circulating electrolytic cell is 25-100° C., a pressure is 0.1-1 MPa, the anion exchange membrane is alcohol-resistant, and the electrolyte at the anode and the electrolyte at the cathode are circulated in a device by the pump.

8. The low energy consumption electrocatalytic method for the electrosynthesis of the hydrogen peroxide coupled with the oxidation upcycling of the PET plastics according to claim 1, wherein in the step (3), the anode electrolyte is adjusted to pH 3-4 by the formic acid, and the terephthalic acid is precipitated.

9. The low energy consumption electrocatalytic method for the electrosynthesis of the hydrogen peroxide coupled with the oxidation upcycling of the PET plastics according to claim 1, wherein in the method 1 of the step (4), a temperature of the ice water bath is 2-10° C. and a reaction time is 1-10 h when the sodium perborate product is prepared.

10. The low energy consumption electrocatalytic method for the electrosynthesis of the hydrogen peroxide coupled with the oxidation upcycling of the PET plastics according to claim 1, wherein in the method 2 of the step (4), a temperature of the second water bath is 5-15° C. and a reaction time is 1-10 h when the benzoyl peroxide is prepared.
